(19) United States
(12) Patent Application Publication
(10) Pub. No.: US 2017/0164899 A1
(43) Pub. Date: Jun. 15, 2017

(54) DEVICES EMBEDDED SMART SHOES

(71) Applicants: ERIKA YANG, LIVERMORE, CA (US); EDWIN MUI, SAN RAMON, CA (US)

(72) Inventors: ERIKA YANG, LIVERMORE, CA (US); EDWIN MUI, SAN RAMON, CA (US)

(21) Appl. No.: 15/343,529

(22) Filed: Nov. 4, 2016

Related U.S. Application Data
(60) Provisional application No. 62/266,720, filed on Dec. 14, 2015.

(51) Int. Cl. A61B 5/00; A43B 7/14; A61H 39/04; A43B 13/38; A61B 5/103; A61H 39/02; A43B 3/00; A43B 17/00

(52) U.S. Cl. CPC A61B 5/6807; A43B 3/0005; A43B 7/146; A43B 17/00; A43B 13/38; A61B 5/1038; A61H 39/02; A61H 39/04; A61B 2562/0271; A61B 2562/12

(57) ABSTRACT

The embodiments disclose a new type of smart shoe with sensor device embedded. The electron signals are collected from the sensors located at the acupuncture points. Through these points, the shoes can create an outline of the way a person walks on average, and can then be uploaded onto an app, where it can be examined by podiatrists. Afterwards, a podiatrist can then determine the best padding to insert into the shoes in order to slowly correct the person's feet and cause their health to increase. In addition, the padding can also be automatically adjusted through the app, which can control the sensors in the padding of the shoe. These types of shoes can be manufactured by 3D printing technology.

### CROSS-REFERENCE TO RELATED APPLICATIONS

The present application claims priority of the provisional application No. 62/266,720 filed on Dec. 14, 2015, the entire contents of which application are incorporated herein for all purposes by this reference.

### FIELD OF THE INVENTION

The present invention generally relates to shoes and methods of using the shoes. More particularly, the present invention relates to devices embedded smart shoes and methods of using the devices embedded smart shoes.

### BACKGROUND

A shoe is an item of footwear intended to protect and comfort the human foot while doing various activities. Shoes are also used as an item of decoration. However, currently there is no shoe that is embedded with smart devices that will improve people's health conditions while walking.

Given the current state of the art, there remains a need for devices embedded smart shoes and methods of using the devices embedded smart shoes that address the abovementioned issues.

The information disclosed in this Background section is provided for an understanding of the general background of the invention and is not an acknowledgement or suggestion that this information forms part of the prior art already known to a person skilled in the art.

### SUMMARY

The embodiments disclose a new type of smart shoe with sensor device embedded. The electron signals are collected from the sensors located at the acupuncture points. Through these points, the shoes can create an outline of the way a person walks on average, and can then be uploaded onto an app, where it can be examined by podiatrists. Afterwards, a podiatrist can then determine the best padding to insert into the shoes in order to slowly correct the person's feet and cause their health to increase. In addition, the padding can also be automatically adjusted through the app, which can control the sensors in the padding of the shoe.

Exemplary systems of the present invention have other features and advantages that will be apparent from or are set forth in more detail in the accompanying drawings, which are incorporated herein, and the following Detailed Description, which together serve to explain certain principles of exemplary embodiments of the present invention.

### BRIEF DESCRIPTION OF THE DRAWINGS

The accompanying drawings, which are incorporated into and constitute a part of this specification, illustrate one or more embodiments of the present application and, together with the detailed description, serve to explain the principles and implementations of the application.

FIG. 1A is a schematic diagram illustrating a devices-embedded smart shoe in accordance with some exemplary embodiments of the present invention. There is a bottom layer called “smart shoe pad (SSP)” in each shoe. The SSP can be either a separated layer or a built-in layer in a shoe.

FIG. 1B a schematic diagram illustrating the SSP, in which there are electronic devices such as two types of electronic devices embedded. One type of electronic devices is the small spot sensors, and the other type of devices is the center control device. The function of the small spot sensor is to detect the signals from the local spot of the foot, such as the movement, pressure, hardness, and etc., and then sends these signals to the center control device for further signal processing.

FIG. 1C a schematic diagram illustrating that the center control device in the SSP will load the signals into the personal communication devices, such as smart tablet devices or display in a screen via Bluetooth internet connection.

FIG. 2A shows a foot with a number of acupuncture points. The acupuncture points have been defined by the medical professionals as points that are connected to the human body's important organs, such as intestine, heart, liver, eyes, ears, and so on. Health status can be improved by massaging these specific acupuncture points.

FIG. 2B shows the positions where the smart shoe pad will specifically hit with many spot sensors, which corresponds to the acupuncture points as shown in FIG. 2A. For example, a spot sensor will be placed in the smart shoe pad where there is an important acupuncture point that connects to an important organ, such as intestine, heart, liver, eyes, ears, and so on, helping to massage the area and improve the overall health.

### DETAILED DESCRIPTION

Reference will now be made in detail to implementations of the exemplary embodiments of the present invention as illustrated in the accompanying drawings. The same reference indicators will be used throughout the drawings and the following detailed description to refer to the same or like parts. Those of ordinary skill in the art will understand that the following detailed description is illustrative only and is not intended to be in any way limiting. Other embodiments of the present invention will readily suggest themselves to such skilled persons having benefit of this disclosure.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Many modifications and variations of the embodiments set forth in this disclosure can be made without departing from their spirit and scope, as will be apparent to those skilled in the art. The specific embodiments described herein are offered by way of example only, and the disclosure is to be limited only by the terms of the appended claims, along with the full scope of equivalents to which such claims are entitled.

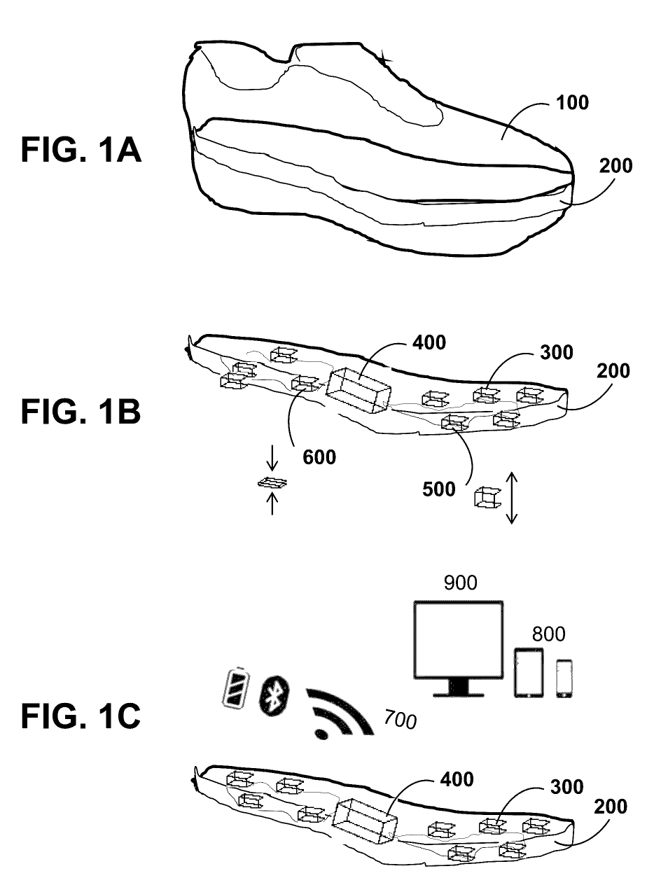

FIG. 1A is a schematic diagram illustrating a devices-embedded smart shoe 100 in accordance with some exemplary embodiments of the present invention. There is a bottom layer 200 called “smart shoe pad (SSP)” in each shoe. The SSP can be either a separated layer or a built-in layer in a shoe.

FIG. 1B a schematic diagram illustrating the SSP 200, in which there are electronic devices such as two types of electronic devices embedded. One type of electronic devices is the small spot sensors 300, and the other type of devices is the center control device 400. The function of the small spot sensor 300 is to detect the signals from the local spot of the foot, such as the movement, pressure, hardness, and etc., and then sends these signals to the center device 400 for further signal processing. Based on the need, the center control device 400 can automatically adjust some acupuncture point area's parameters through a smart device, such as smart phone app. For example, the spot around one acupuncture point 500 needs to expend, whereas the spot around the other acupuncture point 600 needs to be compressed.

FIG. 1C a schematic diagram illustrating that the center control device 400 in the SSP will load the signals into the personal communication devices, such as smart tablet devices 800 or display in a screen 900 via Bluetooth internet connection 700. Signals collected from all spot sensors 300 are sent to the center device 400. The center control device is wirelessly connected to a smart tablet 800 or a screen 900 through Bluetooth 700 and an app.

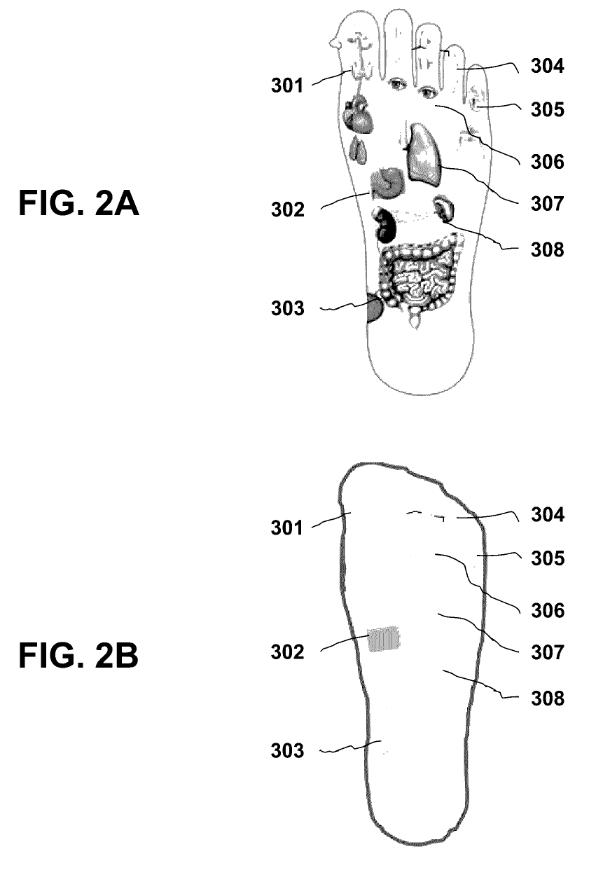

FIG. 2A shows a foot with a number of acupuncture points marked 301-308. The acupuncture points have been defined by the medical professionals as points that are connected to the human body's important organs, such as intestine, heart, liver, eyes, ears, and so on. It has been proven that health status can be improved by massaging these specific acupuncture points.

FIG. 2B shows the positions where the smart shoe pad will specifically hit with many spot sensors, which corresponds to the acupuncture points as shown in FIG. 2A. For example, a spot sensor will be placed in the smart shoe pad where there is an important acupuncture point that connects to an important organ, intestine, heart, liver, eyes, ears, and so on, helping to massage the area and improve the overall health. In a following description, reference is made to the accompanying drawings, which form a part hereof, and in which is shown by way of illustration a specific example in which the invention may be practiced. It is to be understood that other embodiments may be utilized and structural changes may be made without departing from the scope of the present invention.

It should be noted that the descriptions that follow, for example, in terms of smart shoe pad is described for illustrative purposes and the underlying system can apply to any number and multiple type's sensors. In one embodiment of the present invention, a smart shoe pad may include a variety of electronic, optoelectronic and thermal sensors.

These small spot sensors are embedded in the smart shoe. They are located in the locations of the acupuncture points 301-308. The acupuncture points are defined by the medical professionals.

A spot sensor will be placed in the smart shoe pad where there is an important acupuncture point that connects to an important organ, helping to massage the area and improve the overall health.

These small spot sensors 300 embedded in the smart shoe are connected to the center control device 400. The small spot sensors will collect data locally, and then send them to the center control device 400 for further signal processing.

While walking with wearing a smart shoe 100, the center control device 400 will correct the data from all small spot sensors that are placed in the several acupuncture points, and then load signals into the personal communication devices, such as smart tablet devices 800 and screen 900. These collected data from the smart shoe 100 can create an outline of the way a person walks on average and reflect the walker body's health status by making an imprint of the way the person walks.

Based on health status and recommendations from a podiatrist. A customized smart pad shoe (SPS) 710 for a pair of shoes will be made that will best meet the individual person's health needs.

The center control device can automatically adjust some acupuncture point area's parameters through a smart device, such as smart phone app. For example, the spot around one acupuncture point 500 (as shown in FIG. 1B) needs to expend, whereas the spot around the other acupuncture point 600 needs to be compressed. Again, these changes will be performed by software using a smart device.

The changes of shoes or pads include, but not limit to geometry shape, material thickness, material hardness, temperature, and so on. The purpose is to improve person health.

The smart shoe pads or smart shoes can be manufactured by 3D printing technology.

FIG. 1B shows a diagram of the SSP 200, in which there are two types of electronic devices embedded. One type of electronic devices is the small spot sensors 300, and the other type of device is the center control device 400. The function of the small spot sensor 300 is to detect the signals from the local spot of the foot, such as the movement, pressure, hardness, and etc., and then sends these signals to the center device 400 for further signal processing.

Signals collected from all spot sensors 300 are sent to the center device 400. The center control device is wirelessly connected to a smart tablet 800 or a screen 900 through Bluetooth 700 and an app.

FIG. 2A shows a foot with a number of acupuncture points marked 301-308. The acupuncture points have been defined by the medical professionals as points that are connected to the human body's important organs, such as intestine, heart, liver, eyes, ears, and so on. Health status can be improved by massaging these specific acupuncture points.

FIG. 2B shows the positions where the smart shoe pad will specifically hit with many spot sensors, which corresponds to the acupuncture points as shown in FIG. 2A. For example, a spot sensor will be placed in the smart shoe pad where there is an important acupuncture point that connects to an important organ, intestine, heart, liver, eyes, ears, and so on, helping to massage the area and improve the overall health. These small spot sensors are embedded in the smart shoe. They are located in the locations of the acupuncture points 301-308. The acupuncture points are defined by the medical professionals.

A spot sensor will be placed in the smart shoe pad where there is an important acupuncture point that connects to an important organ, helping to massage the area and improve the overall health.

These small spot sensors 300 embedded in the smart shoe are connected to the center control device 400. The small spot sensors will collect data locally, and then send them to the center control device 400 further signal processing.

While walking with wearing a smart shoe 100, the center control device 400 will correct the data from all small spot sensors that are placed in the several acupuncture points, and then load signals into the personal communication devices, such as smart tablet devices 800 and screen 900.

These collected data from the smart shoe 100 can create an outline of the way a person walks on average and reflect the walker body's health status by making an imprint of the way the person walks.

Based on health status and recommendations from a podiatrist. A customized smart pad shoe (SPS) 710 for a pair of shoes will be made that will best meet the individual person's health needs.

The center control device can automatically adjust some acupuncture point area's parameters through a smart device, such as smart phone app. For example, the spot around one acupuncture point 500 (as shown in FIG. 1B) needs to expend, whereas the spot around the other acupuncture point 600 needs to be compressed. Again, these changes will be performed by software using a smart device.

The changes of shoes or pads include, but not limit to geometry shape, material thickness, material hardness, temperature, and so on. The purpose is to improve person health.

The smart shoe pads or smart shoes can be made using a 3-D printer.

The foregoing has described the principles, embodiments and modes of operation of the present invention. However, the invention should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

What is claimed is:

1. A smart shoe 100, comprising:
a smart shoe pad (SSP) 200; and
electronic devices embedded in the SSP, and comprising a plurality of small spot sensors 300 and a center control device 400, wherein:
each small spot sensor is embedded in an acupuncture point for detecting a signal corresponding to a user's foot and sending the signal to the center device for further signals processing.
signals will be sent the shoes can create an outline of the way a person walks on average, and can then be uploaded onto an app.

2. The method of claim 1, further comprising that signals collected from all spot sensors 300 are sent to the center device 400 for further signal processing. The center control device is wirelessly connected to a smart tablet 800 or a screen 900 through Bluetooth 700 and an app

3. The method of claim 1, further comprising a foot with a number of acupuncture points marked 301-308 in FIG. 2A. The acupuncture points have been defined by the medical professionals as points that are connected to the human body's important organs, such as intestine, heart, liver, eyes, ears, and so on. Health status can be improved by massaging these specific acupuncture points.

4. The method of claim 1, further comprising the positions where the smart shoe pad will specifically hit with many spot sensors, which corresponds to the acupuncture points as shown in FIG. 2A. For example, a spot sensor will be placed in the smart shoe pad where there is an important acupuncture point that connects to an important organ, intestine, heart, liver, eyes, ears, and so on, helping to massage the area and improve the overall health.

5. The method of claim 1, further comprising that these small spot sensors are embedded in the smart shoe. They are located in the locations of the acupuncture points 301-308 as shown in FIG. 2B. The acupuncture points are defined by the medical professionals.

6. The method of claim 1, further comprising that a spot sensor will be placed in the smart shoe pad where there is an important acupuncture point that connects to an important organ, helping to massage the area and improve the overall health.

7. The method of claim 1, further comprising that these small spot sensors 300 embedded in the smart shoe are connected to the center control device 400. The small spot sensors will collect data locally, and then send them to the center control device 400 further signal processing.

8. The method of claim 1, further comprising that while walking with wearing a smart shoe 100, the center control device 400 will correct the data from all small spot sensors that are placed in the several acupuncture points, and then load signals into the personal communication devices, such as smart tablet devices 800 and screen 900.

9. The method of claim 1, further comprising that these collected data from the smart shoe 100 can create an outline of the way a person walks on average and reflect the walker body's health status by making an imprint of the way the person walks.

10. The method of claim 1, further comprising that based on health status and recommendations from a podiatrist. A customized smart pad shoe (SPS) 710 for a pair of shoes will be made that will best meet the individual person's health needs.

11. The method of claim 1, further comprising that the center control device can automatically adjust some acupuncture point area's parameters through a smart device, such as smart phone app. For example, the spot around one acupuncture point 500 (as shown in FIG. 1B) needs to expend, whereas the spot around the other acupuncture point 600 needs to be compressed. Again, these changes will be performed by software using a smart device.

12. The method of claim 1, further comprising that the changes of shoes or pads include, but not limit to geometry shape, material thickness, material hardness, temperature, and so on. The purpose is to improve person health.

13. The method of claim 1, further comprising that the smart shoe pads or smart shoes can be manufactured by 3D printing technology.

14. The method of claim 1, further comprising that the small spot sensor can be, but not limited to a variety of electronic sensor, such as optoelectronic sensor, optoelectronic sensor, and thermal sensor.
